(19) United States
(12) Patent Application Publication
(10) Pub. No.: US 2025/0011276 A1
(43) Pub. Date: Jan. 9, 2025

(54) PROCESS FOR THE PREPARATION OF 1,3-BIS(ALLYLAMINO)PROPANE

(71) Applicant: Renosis, Inc., Southlake, TX (US)

(72) Inventors: Wilhelm STAHL, South San Francisco, CA (US); Brian KIRSHNER, South San Francisco, CA (US); Mark JOHNSON, South San Francisco, CA (US); Paul H. KIERSTEAD, South San Francisco, CA (US)

(21) Appl. No.: 17/962,665

(22) Filed: Oct. 10, 2022

Related U.S. Application Data
(63) Continuation of application No. PCT/US2021/028075, filed on Apr. 20, 2021.
(60) Provisional application No. 62/704,085, filed on Apr. 20, 2020.

(51) Int. Cl. C07C 211/22

(52) U.S. Cl. CPC C07C 211/22

(57) ABSTRACT

A process for the preparation of 1,3-bis(allylamino)propane is provided. According to one aspect, the process for the preparation of 1,3-bis(allylamino)propane includes i) forming a reaction mixture containing allylamine (CH₂═CH₂CH₂NH₂) and a difunctional propane, (ii) separating a 1,3-bis(allylamino)propane product from the reaction mixture, and (iii) recovering any excess, unreacted allylamine from the reaction mixture.

### CROSS-REFERENCE TO RELATED APPLICATIONS

This application is a continuation of International Application Number PCT/US2021/028075, filed Apr. 20, 2021, which claims priority to U.S. Patent Application Ser. No. 62/704,085, filed Apr. 20, 2020, the disclosure of each of which is hereby incorporated by reference herein in its entirety.

The present disclosure generally relates to processes for the preparation of 1,3-bis(allylamino)propane.

In US Patent Publication No. 2010/0189679, Inoue et al. disclose the preparation of crosslinked polyallylamine polymers and the acid addition salts thereof via a copolymerization process in which one of the monomers for the copolymerization is an acid addition salt of 1,3-bis(allylamino)propane corresponding to Formula 1:

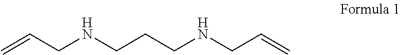

More specifically, Inoue et al. disclose copolymerization of allylammonium dihydrogen phosphate with an acid addition salt of 1,3-bis(allylamino)propane in an amount of 5 to 25 mol % with respect to the amount of said allylammonium dihydrogen phosphate to form a crosslinked polyallylamine polymer or an acid addition salt thereof. Inoue et al. report that the resulting copolymers may be used in the treatment of medical conditions such as hyperphosphatemia.

In U.S. Pat. No. 9,205,107, Klaerner et al. also disclose the preparation of certain crosslinked polyallylamine polymers from 1,3-bis(allylamino)propane or a salt thereof. More specifically, Klaerner et al. disclose copolymerization of allylamine or an acid addition salt thereof, with 1,3-bis(allylamino)propane or an acid addition salt thereof to form a crosslinked polyallylamine polymer. Klaerner et al. report that the resulting copolymers may be used in the treatment of medical conditions such as metabolic acidosis.

In view of the potential use of 1,3-bis(allylamino)propane in the preparation of crosslinked polyallylamine polymers having utility in the treatment of certain medical conditions, a need exists for a relatively efficient and low-cost method for its preparation.

Among the various aspects of the present invention, therefore, may be noted a process for the preparation of 1,3-bis(allylamino)propane or a salt thereof.

In one embodiment, the process of the present disclosure comprises the preparation of 1,3-bis(allylamino)propane or a salt thereof from allylamine and a difunctional propane having the structural formula XCH₂CH₂CH₂X wherein each X is independently halo, tosylate, mesylate, triflate, acetate, or triflouoroacetate. For example, in one such embodiment, the difunctional propane is a 1,3-dihalopropane. By way of further example, in one such embodiment, the difunctional propane is 1,3-dichloropropane.

In one embodiment, the process of the present disclosure comprises the preparation of 1,3-bis(allylamino)propane or a salt thereof from allylamine and a difunctional propane in a reaction mixture in which there is an excess of allylamine relative to the difunctional propane, and upon completion of the reaction to the desired degree, 1,3-bis(allylamino)propane product is separated from the reaction mixture and the excess, unreacted allylamine is recycled for reuse.

Other aspects and features will be in part apparent and in part pointed out hereinafter.

### BRIEF DESCRIPTION OF THE DRAWINGS

FIG. 1 is a flow chart schematically depicting a process for the preparation of 1,3-bis(allylamino)propane in accordance with a first embodiment of the present disclosure.

FIG. 2 is a flow chart schematically depicting a process for the preparation of 1,3-bis(allylamino)propane in accordance with a second embodiment of the present disclosure.

FIG. 3 is a flow chart schematically depicting a process for the preparation of 1,3-bis(allylamino)propane in accordance with a third embodiment of the present disclosure.

FIG. 4 is a flow chart schematically depicting a process for the preparation of 1,3-bis(allylamino)propane in accordance with a fourth embodiment of the present disclosure.

FIG. 5 is a flow chart schematically depicting a process for the preparation of 1,3-bis(allylamino)propane in accordance with a fifth embodiment of the present disclosure.

FIG. 6 is a flow chart schematically depicting a process for the preparation of 1,3-bis(allylamino)propane in accordance with a sixth embodiment of the present disclosure.

### ABBREVIATIONS AND DEFINITIONS

The following definitions and methods are provided to better define the present invention and to guide those of ordinary skill in the art in the practice of the present invention. Unless otherwise noted, terms are to be understood according to conventional usage by those of ordinary skill in the relevant art.

The term “acetate” as used herein refers to a moiety having the structural formula *—OC(═O)CH₃, where * denotes the point of attachment of the moiety to the remainder of the molecule.

The term “allyl” denotes a moiety having the structural formula H₂C═CH—CH₂—*, where * denotes the point of attachment of the moiety to the remainder of the molecule and the point of attachment is to a heteroatom or an aromatic moiety.

The term “allylamine” as used herein denotes a moiety having the structural formula H₂C═CH—CH₂NH₂.

The term “amine” or “amino” as used alone or as part of another group, represents a group of formula ·—N(X₈)(X₉), wherein X₈ and X₉ are independently hydrogen, hydrocarbyl, or substituted hydrocarbyl, heteroaryl, or heterocyclo, or X₈ and X₉ taken together form a substituted or unsubstituted alicyclic, aryl, or heterocyclic moiety, each as defined in connection with such term, typically having from 3 to 8 atoms in the ring, and * denotes the point of attachment of the moiety to the remainder of the molecule.

The term “1,3-bis(allylamino)propane” denotes a moiety having the structural formula CH₂═CH₂CH₂NHCH₂CH₂CH₂NHCH₂CH₂═CH₂, corresponding to Formula 1. It is also sometimes referred to as N,N′-diallyl-1,3-diaminopropane, diallylpropyldiamine, or more simply, by the acronym “DAPDA” or the acronym “DAAH”.

The term “1,3-bis(allylamino)propane product” as used herein includes 1,3-bis(allylamino)propane in its free base form and in the form of any acid addition salt thereof that may be recovered as a product from the reaction mixtures described herein.

The term “difunctional propane” as used herein means a propyl moiety having the structural formula XCH₂CH₂CH₂X wherein each X is independently halo, tosylate, mesylate, triflate, acetate, or triflouoroacetate. In certain currently preferred embodiments, the difunctional propane is a 1,3-dihalopropane.

The term “1,3-dihalopropane” as used herein means a difunctional propane having the structural formula XCH₂CH₂CH₂X wherein each X is independently selected from the group consisting chloro, fluoro, bromo, and iodo. In certain currently preferred embodiments, the difunctional propane is 1,3-chloropropane.

The term “halo” means halogens such as fluorine, chlorine, bromine or iodine atoms.

The term “mesylate” as used herein refers to a moiety having the structural formula ·—OSO₂CH₃, where * denotes the point of attachment of the moiety to the remainder of the molecule.

The terms “optional” and “optionally” mean that the subsequently described event or circumstance may but need not occur, and that the description includes instances where the event or circumstance occurs and instances in which it does not.

The term “tosylate” as used herein refers to a moiety having the structural formula ·—OSO₂C₇H₇, where * denotes the point of attachment of the moiety to the remainder of the molecule.

The term “triflate” as used herein refers to a moiety having the structural formula ·—OSO₂CF₃, where * denotes the point of attachment of the moiety to the remainder of the molecule.

The term “trifluoroacetate” as used herein refers to a moiety having the structural formula ·—OSO₂CF₃, where * denotes the point of attachment of the moiety to the remainder of the molecule.

When introducing elements of the present invention or the preferred embodiments(s) thereof, the articles “a”, “an”, “the” and “said” are intended to mean that there are one or more of the elements. The terms “comprising”, “including” and “having” are intended to be inclusive and not exclusive (i.e., there may be other elements in addition to the recited elements).

### EMBODIMENTS

In accordance with the present disclosure, 1,3-bis(allylamino)propane or an acid addition salt thereof may be prepared in a reaction mixture containing (i) allylamine (CH₂═CH₂CH₂NH₂) and (ii) a difunctional propane having the structural formula XCH₂CH₂CH₂X wherein each X is independently halo, tosylate, mesylate, triflate, acetate, or triflouoroacetate. For example, in one such embodiment, the hydrochloride salt of 1,3-bis(allylamino)propane may be prepared in a reaction mixture containing allylamine (CH₂═CH₂CH₂NH₂) and 1,3-dichloropropane, as illustrated in Reaction Scheme 1:

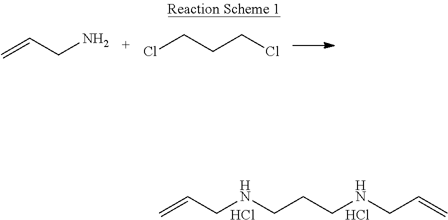

In general, the process comprises (i) forming a reaction mixture containing allylamine (CH₂=CH₂CH₂NH₂) and a difunctional propane, (ii) separating the 1,3-bis(allylamino)propane product from the reaction mixture, and (iii) recovering any excess, unreacted allylamine from the reaction mixture. As described in greater detail elsewhere herein, the process may be carried out in a batch, semi-continuous or continuous mode.

The reaction between the allylamine and difunctional propane may be run neat, that is, the reaction is carried out in the liquid phase in the absence of any solvent. Alternatively, the reaction mixture may contain a solvent system comprising a solvent such as acetonitrile, carbon tetrachloride, dichloromethane, diethylether, dimethylcarbonate, N,N-dimethylformamide, dimethylsulfoxide, tetrahydrofuran, or a combination thereof. In those embodiments in which the reaction mixture includes a solvent, the solvent may be introduced to the reaction mixture by any of several approaches. For example, (i) the solvent may be combined with allylamine and the resulting allylamine-solvent mixture is introduced to the reaction mixture, (ii) the solvent may be combined with the difunctional propane and the resulting difunctional propane-solvent mixture is introduced to the reaction mixture, and/or (iii) the solvent may be introduced to the reaction mixture independent of the allylamine and the difunctional propane reactants.

In general, it is preferred that the reaction mixture contain an excess of allylamine relative to the difunctional propane. For example, in some embodiments it is preferred that the molar ratio of allylamine to difunctional propane in the reaction mixture be at least about 2.5:1, respectively. By way of further example, in some embodiments it is preferred that the molar ratio of allylamine to difunctional propane in the reaction mixture be at least about 5:1, respectively. By way of further example, in some embodiments it is preferred that the molar ratio of allylamine to difunctional propane in the reaction mixture be at least about 15:1, respectively. In general, however, it is typically preferred that the molar ratio of allylamine to difunctional propane in the reaction mixture be less than about 25:1, respectively. To limit the occurrence of multiple reactions on the amine group of allylamine, the difunctional propane may be slowly metered into a solution containing allylamine or neat allylamine. This, in effect, results in a very large excess of allylamine in the mixture at any given time, as the difunctional propane is consumed by reaction as it is added. By limiting the amount of difunctional propane present at any given time, over-substitution of the amine group is limited.

Although not presently preferred, in certain embodiments the reaction mixture may optionally contain any of a range of other compositions to aid the reaction. For example, the reaction mixture may optionally contain an additive/catalyst for this type of reaction such as an iodide salt (e.g., sodium iodide or potassium iodide). Alternatively, a variety of non-nucleophilic bases such as N-diisopropylethylamine, 1,8-diazabicycloundec-7-ene, lithium diisopropylamide, sodium or potassium hydride, sodium or potassium tert-butoxide, or sodium bis(trimethylsilyl)amide) could alternatively be included.

As described in greater detail elsewhere herein, the reaction between the allylamine and difunctional propane reactants may be carried out in any of a variety of chemical reactor vessels, e.g., a batch reactor, a continuous stirred tank reactor, a plug flow reactor, or a continuous distillation column reactor/separator. Depending upon the reactor vessel type, the reaction mixture may be advantageously formed in the reactor vessel, i.e., the allylamine and difunctional propane reactants (and any additional optional compositions) may be separately introduced to the reactor vessel to form the reaction mixture, in situ, or alternatively, the allylamine and difunctional propane reactants (and any additional optional compositions) may be combined outside the reactor vessel to form the reaction mixture and the resulting reaction mixture is then introduced to the reactor vessel.

As the reaction between the allylamine and difunctional propane reactants proceeds in the reactor vessel, the reaction mixture will be maintained in an atmosphere and at a temperature and pressure conducive to the formation of the 1,3-bis(allylamino)propane product, taking into account a range of factors including, for example, the reaction reactor vessel type, the composition of the reaction mixture, and the target reaction kinetics and product profile. In general, however, the reaction will typically be allowed to proceed in an inert atmosphere, e.g., nitrogen, argon, helium, carbon dioxide, or a mixture thereof. Additionally, the reaction mixture will typically be maintained in the reactor vessel at a pressure in the range of about 0.8 to about 2 atm, and at a temperature within the range of about 50° C. to about 100° C. until the desired degree of conversion of the reactants to the 1,3-bis(allylamino)propane product is achieved.

Once the desired degree of conversion of the reactants to the 1,3-bis(allylamino)propane product is achieved, the 1,3-bis(allylamino)propane product, for example, as the di-HCl salt, may be separated from the reaction mixture and any excess, unreacted allylamine and any excess unreacted difunctional propane, if any, is preferably recovered and recycled for reuse in the absence of a precipitation solvent by means such as continuous belt-filter drying, wiped film evaporation or flash evaporation. If a precipitation solvent is used to separate the product from any unreacted allylamine then a continuous precipitation solvent/allylamine separator can be used to recover and recycle the unreacted allylamine by means such as decantation, liquid-liquid extraction, distillation or centrifugation. Exemplary precipitation solvents include water, isopropanol, n-propanol, methanol, ethanol, butanol, acetone, acetonitrile, ethyl acetate, tetrahydrofuran, dichloromethane, nitromethane, acetic acid, formic acid. These separation techniques can also be used to recover and recycle the precipitation solvent. In one currently preferred embodiment, the 1,3-bis(allylamino)propane product is recovered by distillation. For example, upon reaching the desired degree of conversion, the reaction mixture may be distilled to recover excess allylamine and the recovered allylamine is recycled for re-use as a starting material in a subsequent batch, semi-continuous or continuous reaction; similarly, any excess, unreacted difunctional propane in the reaction mixture upon reaching the desired degree of conversion may be recovered and recycled for use in a subsequent batch, semi-continuous or continuous reaction.

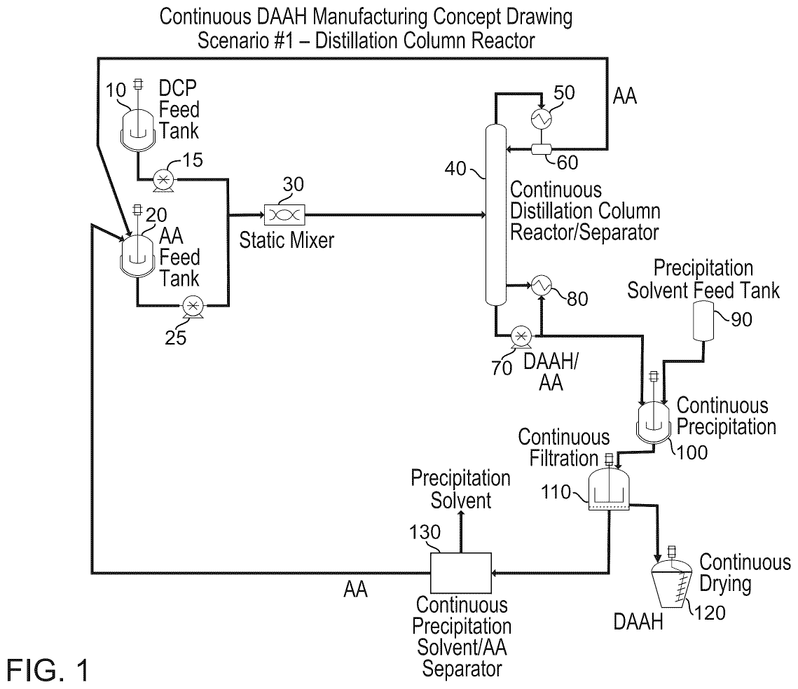

Referring now to FIG. 1 (Scenario #1), in a first alternative exemplary embodiment, difunctional propane, such as dichloropropane (DCP), is fed from DCP feed tank (10) into static mixer (30) with the flow rate controlled by pump 15. Allylamine (AA) is fed from AA feed tank (20) into static mixer (30) with the flow rate controlled by pump 25. After the allylamine and difunctional propane reactants are combined in the static mixture, the reaction mixture is introduced to continuous distillation column reactor/separator (40). In continuous distillation column reactor/separator (40), the allylamine and difunctional propane reactants combine to form the 1,3-bis(allylamino)propane product, with distilled allylamine (AA) being removed from the top of the column and 1,3-bis(allylamino)propane product (DAAH) being removed from the bottom of the column. The distillation column (40) may contain trays, random packing or structured packing and operated, for example, at a temperature in the range of about 50° C. to 150° C., a pressure of about 5 to 30 PSIA, a reflux ratio of about 1:1 to about 10:1, and a bottoms vs overheads removal ratio of about 1:1 to about 8:1 (DCP:AA). Distilled allylamine (AA) is recovered (50, 60) and returned to allylamine feed tank 20. The 1,3-bis(allylamino)propane product (DAAH), is removed (70, 80) and transferred to the continuous precipitation vessel 100 which may be a distillation column, decanter, liquid-liquid extractor, wiped film evaporator or the like. The 1,3-bis(allylamino)propane product (DAAH) is precipitated using a precipitation solvent from tank (90). The 1,3-bis(allylamino)propane product (DAAH) is isolated by continuous filtration in vessel 110 and dried in vessel 120. Allylamine is separated from precipitation solvent in vessel 130, recovered, and returned to allylamine feed tank 20.

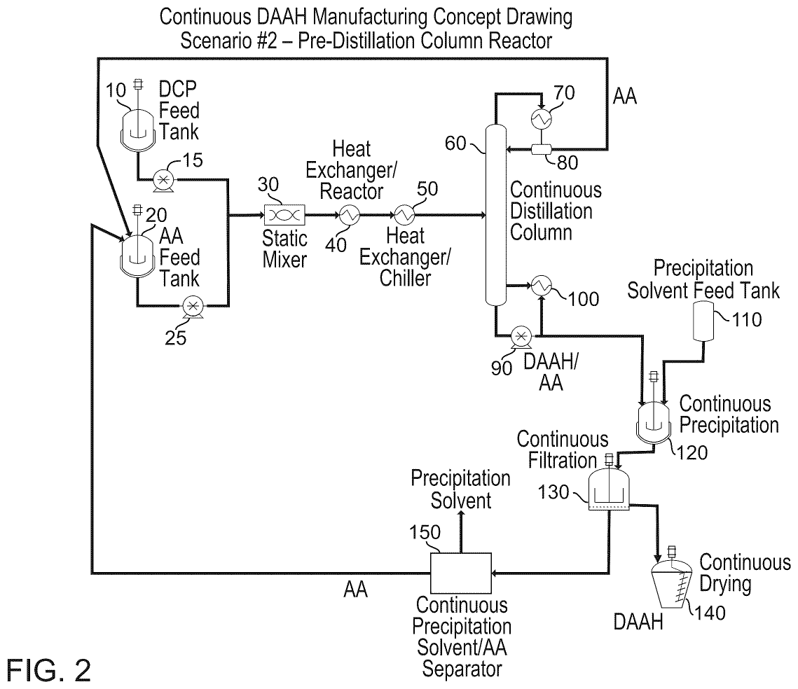

Referring now to FIG. 2 (Scenario #2), in a second alternative exemplary embodiment, dichloropropane (DCP) is fed from DCP feed tank (10) into static mixer/heat exchanger/reactor (30, 40, 50) with the flow rate controlled by pump 15. Allylamine (AA) is fed from AA feed tank (20) into static mixer/heat exchanger/reactor (30, 40, 50) with the flow rate controlled by pump 25. The reaction mixture is introduced to distillation column (60) and the excess allylamine removed. Distilled allylamine is recovered (70, 80) and returned to allylamine feed tank 20. The reaction product is removed (90, 100) and transferred to continuous precipitation vessel 120. The product is precipitated using a precipitation solvent from tank (110). The product is isolated by continuous filtration in vessel 130 and dried in vessel 140. Allylamine is separated from precipitation solvent in vessel 150, recovered, and returned to allylamine feed tank 20.

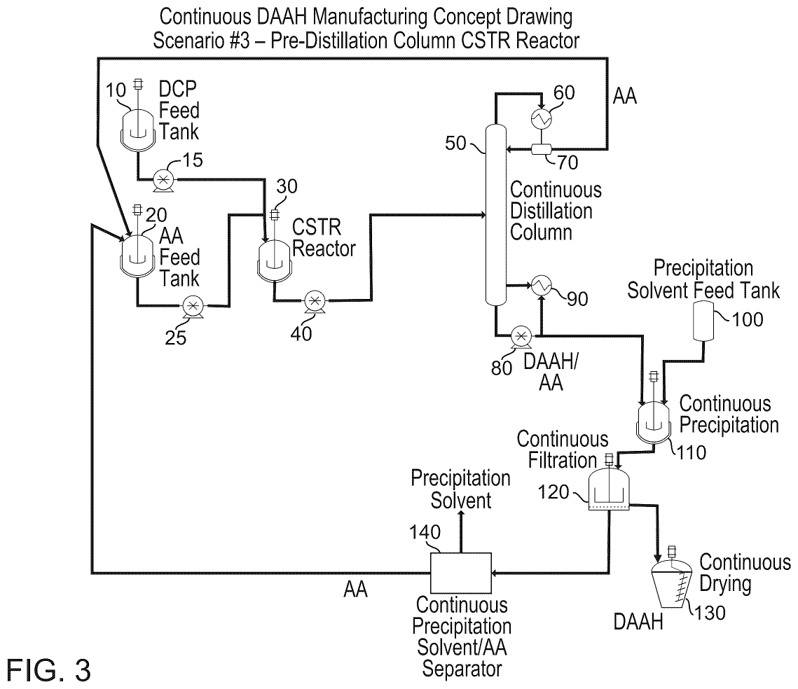

Referring now to FIG. 3 (Scenario #3), in a third exemplary embodiment, dichloropropane (DCP) is fed from DCP feed tank (10) into reactor (30) with the flow rate controlled by pump 15. Allylamine (AA) is fed from AA feed tank (20) into reactor (30) with the flow rate controlled by pump 25. The reaction mixture is fed from reactor (30), introduced to distillation column (50) and the excess allylamine removed. Distilled allylamine is recovered (60, 70) and returned to allylamine feed tank 20. The reaction product is removed (80, 90) and transferred to continuous precipitation vessel 110. The product is precipitated using a precipitation solvent from tank (100). The product is isolated by continuous filtration in vessel 120 and dried in vessel 130. Allylamine is separated from precipitation solvent in vessel 140, recovered, and returned to allylamine feed tank 20.

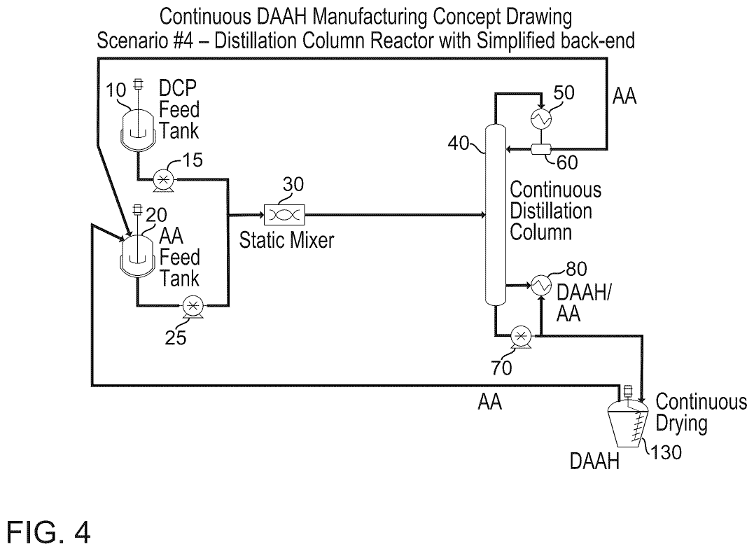

Referring now to FIG. 4 (Scenario #4), in a fourth exemplary embodiment, dichloropropane (DCP) is fed from DCP feed tank (10) into static mixer (30) with the flow rate controlled by pump 15. Allylamine (AA) is fed from AA feed tank (20) into static mixer (30) with the flow rate controlled by pump 25. The reaction mixture is introduced to distillation column (40) and the excess allylamine removed. Distilled allylamine is recovered (50, 60) and returned to allylamine feed tank 20. The reaction product is removed (70, 80) and transferred to continuous dryer 130. Allylamine is separated from DAAH in the dryer 130 and is returned to allylamine feed tank 20. The process of the current scenario, i.e., Scenario #4, is the same as the process of Scenario #1 except that neither a precipitation solvent feed tank, nor a continuous precipitator, a continuous filter, a continuous precipitation solvent/allylamine separator are used in this scenario.

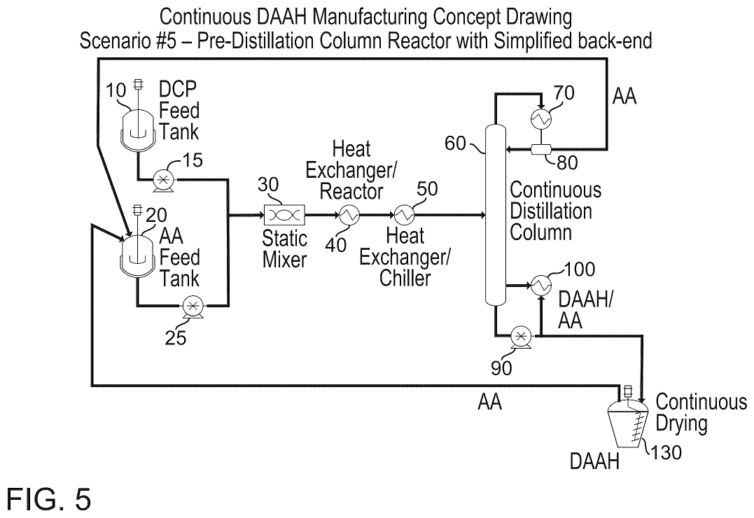

Referring now to FIG. 5 (Scenario #5), in a fifth exemplary embodiment, dichloropropane (DCP) is fed from DCP feed tank (10) into static mixer/heat exchanger/reactor (30, 40, 50) with the flow rate controlled by pump 15. Allylamine (AA) is fed from AA feed tank (20) into static mixer/heat exchanger/reactor (30, 40, 50) with the flow rate controlled by pump 25. The reaction mixture is introduced to distillation column (60) and the excess allylamine removed. Distilled allylamine is recovered (70, 80) and returned to allylamine feed tank 20. The reaction product is removed (90, 100) and transferred to continuous dryer 130. Allylamine is separated from DAAH in the dryer 130 and is returned to allylamine feed tank 20. The process of the current scenario, i.e., Scenario #5, is the same as the process of Scenario #2 except that neither a precipitation solvent feed tank, nor a continuous precipitator, a continuous filter, a continuous precipitation solvent/allylamine separator are used in this scenario.

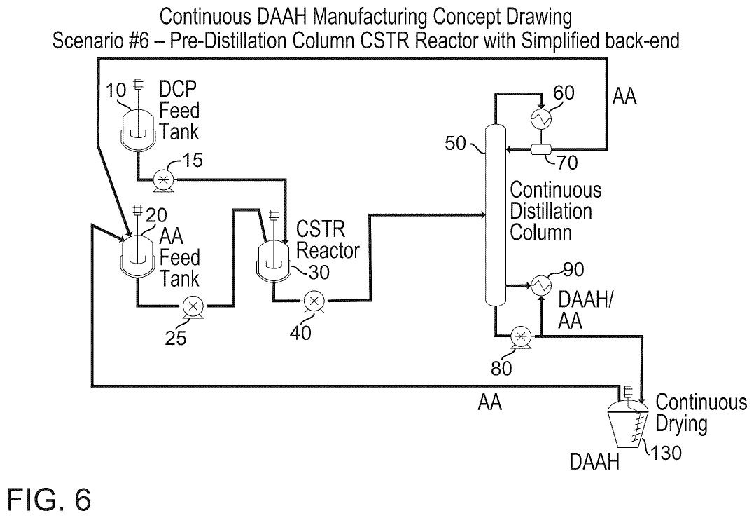

Referring now to FIG. 6 (Scenario #6), in a sixth exemplary embodiment, dichloropropane (DCP) is fed from DCP feed tank (10) into reactor (30) with the flow rate controlled by pump 15. Allylamine (AA) is fed from AA feed tank (20) into reactor (30) with the flow rate controlled by pump 25. The reaction mixture is fed from reactor (30), introduced to distillation column (50) and the excess allylamine removed. Distilled allylamine is recovered (60, 70) and returned to allylamine feed tank 20. The reaction product is removed (80, 90) and transferred to continuous dryer 130. Allylamine is separated from DAAH in the dryer 130 and is returned to allylamine feed tank 20. The process of the current scenario, i.e., Scenario #6, is the same as the process of Scenario #3 except that neither a precipitation solvent feed tank, nor a continuous precipitator, a continuous filter, a continuous precipitation solvent/allylamine separator are used in this scenario.

In further embodiments, enumerated as embodiments 1-10 below, the present disclosure includes:

Embodiment 1. A process for the preparation of 1,3-bis(allylamino)propane, the process comprising i) forming a reaction mixture containing allylamine (CH₂=CH₂CH₂NH₂) and a difunctional propane, (ii) separating a 1,3-bis(allylamino)propane product from the reaction mixture, and (iii) recovering any excess, unreacted allylamine from the reaction mixture.

Embodiment 2. The process of Embodiment 1 wherein the process is carried out as a batch process.

Embodiment 3. The process of Embodiment 1 wherein the process is carried out as a semi-continuous process.

Embodiment 4. The process of Embodiment 1 wherein the process is carried out as a continuous process.

Embodiment 5. The process of any previous enumerated Embodiment wherein the reaction between the allylamine and the difunctional propane is carried out in a reactor vessel selected from the group consisting of a batch reactor, continuous stirred tank reactor, plug flow reactor, or a continuous distillation column reactor/separator.

Embodiment 6. The process of any previous enumerated Embodiment wherein the reaction mixture is formed in the reactor vessel.

Embodiment 7. The process of any previous enumerated Embodiment wherein the allylamine and difunctional propane reactants (and any additional optional compositions) are combined outside the reactor vessel to form the reaction mixture and the resulting reaction mixture is then introduced to the reactor vessel.

Embodiment 8. The process of any previous enumerated Embodiment wherein the 1,3-bis(allylamino)propane product may be separated from the reaction mixture and any excess, unreacted allylamine and/or any excess unreacted difunctional propane, if any, is recovered and recycled for reuse as a starting material in a subsequent batch, semi-continuous or continuous reaction.

Embodiment 9. The process of any previous enumerated Embodiment wherein a precipitation solvent is used to separate the 1,3-bis(allylamino)propane product from the reaction mixture.

Embodiment 10. The process of Embodiment 9 wherein the precipitation solvent is selected from the group consisting of water, isopropanol, n-propanol, methanol, ethanol, butanol, acetone, acetonitrile, ethyl acetate, tetrahydrofuran, dichloromethane, nitromethane, acetic acid, formic acid and combinations thereof.

Having described the invention in detail, it will be apparent that modifications and variations are possible without departing the scope of the invention defined in the appended claims. Furthermore, it should be appreciated that all examples in the present disclosure are provided as non-limiting examples.

### EXAMPLES

The following non-limiting examples are provided to further illustrate the present invention. It should be appreciated by those of skill in the art that the techniques disclosed in the examples that follow represent approaches the inventors have found function well in the practice of the invention, and thus can be considered to constitute examples of modes for its practice. However, those of skill in the art should, in light of the present disclosure, appreciate that many changes can be made in the specific embodiments that are disclosed and still obtain a like or similar result without departing from the spirit and scope of the invention.

### Example 1

1,3-Dichloropropane (80 g, 0.714 mol) and allylamine (400 g, 7.14 mol) were placed into a flask and the resulting mixture was heated with stirring under argon atmosphere at 50 to 52° C. for 20 hours. About a half amount of the excessive allylamine was evaporated under reduced pressure, the residue was washed with i-PrOH, filtered to afford the desired product as a white solid (113 g, 70%). 1H NMR (D₂O, 400 MHZ): δ 5.80-5.98 (m, 2H), 5.50-5.60 (m, 4H), 3.70-3.80 (m, 4H), 3.10-3.20 (d, 4H), 2.02-2.12 (m, 2H).

1. A process for the preparation of 1,3-bis(allylamino)propane, the process comprising i) forming a reaction mixture containing allylamine (CH2=CH2CH2NH2) and a difunctional propane, (ii) separating a 1,3-bis(allylamino)propane product from the reaction mixture, and (iii) recovering any excess, unreacted allylamine from the reaction mixture, wherein the process is selected from the group consisting of a batch process, semi-continuous process, and continuous process.

2-4. (canceled)

5. The process of claim 1, wherein the reaction between the allylamine and the difunctional propane is carried out in a reactor vessel selected from the group consisting of a batch reactor, continuous stirred tank reactor, plug flow reactor, or a continuous distillation column reactor/separator.

6-8. (canceled)

9. The process of claim 1, wherein the reaction mixture is formed in the reactor vessel.

10-16. (canceled)

17. The process of claim 1, wherein at least the allylamine and difunctional propane reactants are combined outside the reactor vessel to form the reaction mixture and the resulting reaction mixture is then introduced to the reactor vessel.

18-32. (canceled)

33. The process of claim 1, wherein the 1,3-bis(allylamino)propane product is separated from the reaction mixture and any excess unreacted allylamine, excess unreacted difunctional propane, or the combination thereof, is recovered and recycled for reuse as a starting material in a subsequent batch, semi-continuous or continuous reaction.

34-64. (canceled)

65. The process of claim 1, wherein a precipitation solvent is used to separate the 1,3-bis(allylamino)propane product from the reaction mixture.

66. The process of claim 65, wherein the precipitation solvent is selected from the group consisting of water, isopropanol, n-propanol, methanol, ethanol, butanol, acetone, acetonitrile, ethyl acetate, tetrahydrofuran, dichloromethane, nitromethane, acetic acid, formic acid and combinations thereof.

67-98. (canceled)
